(19) United States
(12) Patent Application Publication
(10) Pub. No.: US 2006/0229691 A1
(43) Pub. Date: Oct. 12, 2006

(54) System and method of applying a therapeutic treatment

(76) Inventors: Alexander G. Noskov, Rochester Hills, MI (US); Delphine L. Agnor, Rochester Hills, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 11/102,402

(22) Filed: Apr. 8, 2005

(51) Int. Cl. A61F 7/00; A61F 7/12

(52) U.S. Cl. 607/96

(57) ABSTRACT

A system and method for applying a therapeutic treatment to occupants of an enclosure. The enclosure includes at least one treatment area configured to receive and support one or more occupants. One or more heating panels are provided on the at least one treatment area to generate radiant heat effecting therapeutic changes in human body temperature. A ventilation system maintains environmental settings in the enclosure, supplying oxygen to the enclosure and removing sated air from the enclosure. A controller monitors the at least one treatment area and ventilation system to control therapeutic conditions in the enclosure.

### CROSS-REFERENCE TO RELATED APPLICATIONS

This application claims the benefit of U.S. provisional application Ser. No. 60/511,811, filed Sep. 21, 2004.

### BACKGROUND OF THE INVENTION

1. Field of the Invention

The present invention relates to a system and method of applying a therapeutic treatment and more particularly to a system and method for producing a therapeutic effect in the human body by applying electromagnetic energy to increase body temperature.

2. Background Art

The therapeutic effects of human body temperature adjustment have been used by medical and holistic practitioners to treat a variety of ailments. Practitioners have studied the use of thermal heating process as a means of fighting many serious diseases based on the principle that a rise in body temperature higher than 37 degrees Celsius (98.6 degrees Fahrenheit) kills viruses, bacteria, and other microorganisms which do not maintain heat. Increased temperatures further stimulate the body to manufacture antibodies and interferon, which in turn, blocks pathogens from developing. Physiological effects such as cardiovascular and muscular activity, blood circulation and kidney function have all shown improvement through the use of heat therapy.

There are several well known therapeutic treatments in the art, including a Finnish sauna, Infrared Cabin, the Turkish banya, Russian steam bath and stone furnace, which expose humans to increased temperatures. However, while these methods create an increase in body temperature, they do not adequately provide a consistent therapeutic effect and may create harmful side effects.

A Finnish sauna system generates temperatures between 80-120 degrees Celsius (176-248 degrees Fahrenheit) with relatively low humidity. The body is warmed from air heated by a furnace. Rapid evaporation of perspiration from the human body in the dry air of the sauna creates a layer of steam between the skin and the hot air in the sauna. The layer of steam prevents the body from exceeding its normal temperature, provided that the air environment and the person remain motionless.

The Finnish sauna generates extreme temperatures, forcing the human body to combat the outer temperature by perspiration rather than absorbing the beneficial effects of the heat. Thus, the internal temperature of the person, beyond the body's outer surface, is not increased. Further, a person cannot remain in the extreme environment of the Finnish sauna for prolonged periods of time, which may produce unwanted physiological effects.

Infrared cabins, unlike the Finnish sauna, utilize radiant heat rather than heated air to generate the therapeutic effect. High temperature infrared cabins in the range of 300 degrees Celsius (572 degrees Fahrenheit) radiate waves of 2 to 6.5 micrometers, which are absorbed by the surface of the body, the skin and thin layer of hypodermic cellular tissue. The radiant waves are carried through the body by a current of blood from thin hypodermic capillaries and heat conductivity of the cells.

Infrared cabins do not effect a human's temperature beyond the body's surface layer because the body is working to produce perspiration to combat the extreme temperature. Thus, the core internal body temperature is not effected by the treatment. Further, local over-heating in infra red cabins lead to the partial dying off of cells. Infrared waves generated in the 2 to 6.5 micrometer range are known as “hard” radiation which can speed the aging process.

Turkish banyas usually are formed with stone floors and plank beds of varying temperatures utilizing aqueducts to carry hot water or steam. Both air and stone generate infrared radiant heat, with temperatures ranging from 60-90 degrees Celsius (140-194 degrees Fahrenheit) and humidity in excess of 60 percent. This radiant heat does effect the core internal body temperature. However, the benefits of the radiant heat are negated by the high moisture content of the air when humidity exceeds 60 percent.

A Russian steam bath generates air temperatures between 50 and 90 degrees Celsius (122-194 degrees Fahrenheit) and humidity levels of up to 80 percent. Russian steam bath rooms provide plank beds at varying heights. Russian steam baths maximize the inverse relationship between temperature and humidity by varying the amount of steam and temperature produced by casting water upon stones heated in the furnace. The variance of steam content in the Russian steam bath allows for body absorption of infrared radiant heat in combination with the inhalation of the heated damp air.

Russian steam baths may also employ the use of bound leaves to massage the body. Use of the leaves confers acute temperature upon the skin by breaking the temperature barrier between the air and the skin. Although deep heating of the body occurs as a result of the body's absorbing the infrared radiant heat which emanates from the heated brick walls of the steam bath furnace, infrared heating of the human body is not uniform, stunting the overall therapeutic effects of the steam bath.

It would be advantageous to provide a system and method for effecting a change in human body temperature that eliminates the harmful effects of these well known therapy options. It would also be advantageous to provide a system and method which provides deep warming of the internal core temperature of a human body while providing more comfortable conditions, including elimination of perspiration, reduction of stress on the cardiovascular system and inhalation of damp warm air.

It would be further advantageous to provide a system and method for effecting change in human body temperature which uses the body's own immune system coupled with the rise in temperature, to provide tangible benefits to the body. It would also be advantageous to provide a system and method for effecting change in human body temperature by uniformly warming the human body internal core temperature with the use of soft infrared radiant heat generated by igneous rock sources.

### SUMMARY OF THE INVENTION

Accordingly, in accordance with the present invention, a system and method for applying a therapeutic treatment to occupants of an enclosure. The enclosure includes at least one treatment area configured to receive and support one or more occupants. One or more heating panels are provided on the at least one treatment area to generate radiant heat effecting therapeutic changes in human body temperature. A ventilation system maintains environmental settings in the enclosure, supplying oxygen to the enclosure and removing sated air from the enclosure. A controller monitors the at least one treatment area and ventilation system to control therapeutic conditions in the enclosure.

The enclosure comprises a therapy room having a ceiling, a floor, a pair of opposing sidewalls extending therebetween, a forward wall and an opposing rear wall. A seating bench and laying bench are provided in the therapy room. Each bench includes a frame supporting one or more heating panels configured to generate radiant heat effecting therapeutic changes in human body temperature. The heating panels are formed of a frame supporting a heating plate and one or more heating elements disposed between the frame and heating plate. The seating bench is designed to support one or more occupants in a generally upright position while the laying bench supports an occupant in a generally horizontal position.

The above features, and other features and advantages of the present invention are readily apparent from the following detailed descriptions thereof when taken in connection with the accompanying drawings.

### BRIEF DESCRIPTION OF THE DRAWINGS

FIG. 1 is a front perspective view of a therapeutic treatment enclosure for use with the system and method of the present invention;

FIG. 2 is a rear perspective view of the therapeutic treatment enclosure and ventilation system in accordance with the present invention;

FIG. 3 is a perspective view of occupants positioned on a seating bench used in conjunction with the therapeutic treatment enclosure;

FIG. 4 is a cross-sectional view of the seating bench along line 4-4 of FIG. 3;

FIG. 5 is a perspective view of an occupant positioned on a warming table or laying bench used in conjunction with the therapeutic treatment enclosure;

FIG. 6 is a side elevational view of a heating panel configured for use in the therapeutic treatment enclosure;

FIG. 7 is an exploded cross-sectional view of the ceiling panel along line 7-7 of FIG. 6;

FIG. 8 is a perspective view of a sidewall panel for use in the therapeutic treatment enclosure incorporating a lamp;

FIG. 9 is a perspective view of the floor of the therapeutic treatment enclosure; and

FIG. 10 is a perspective view of the therapeutic treatment enclosure having a ventilation system including one or more heat recovery units in accordance with the present invention.

### DETAILED DESCRIPTION OF THE PREFERRED EMBODIMENT(S)

Certain terminology presented below is used for convenience in reference only and is not intended to be limiting. More specifically, directional language such as “left”, “right”, “above”, “below”, “upper”, “lower” and words of similar import designate directions shown in the drawings. Such directional terminology is used for clarity and is not intended to strictly limit the orientation of any aspect of the invention to a particular plane or direction.

As required, detailed embodiments of the present invention are disclosed herein. However, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a representative basis for the claims and/or as a representative basis for teaching one skilled in the art to variously employ the present invention.

Referring now to the Figures, a system and method for applying electromagnetic energy to effect therapeutic changes in human body temperature in a therapeutic treatment enclosure is illustrated and disclosed. FIG. 1 illustrates one aspect of the therapeutic system in accordance with the present invention. A therapeutic enclosure 100 includes a ceiling 1, a pair of opposing sidewalls 2, 4, a back or rear wall 3, an opposing forward or front wall 5 and a floor 21. The therapeutic enclosure is typically an enclosed space such as a room. A heating alcove 7 provided adjacent the rear wall 3 of room 100 incorporates a ventilation system and heating platform 10. The front wall 5 includes a portal or door 6 allowing entry of an occupant into the room 100.

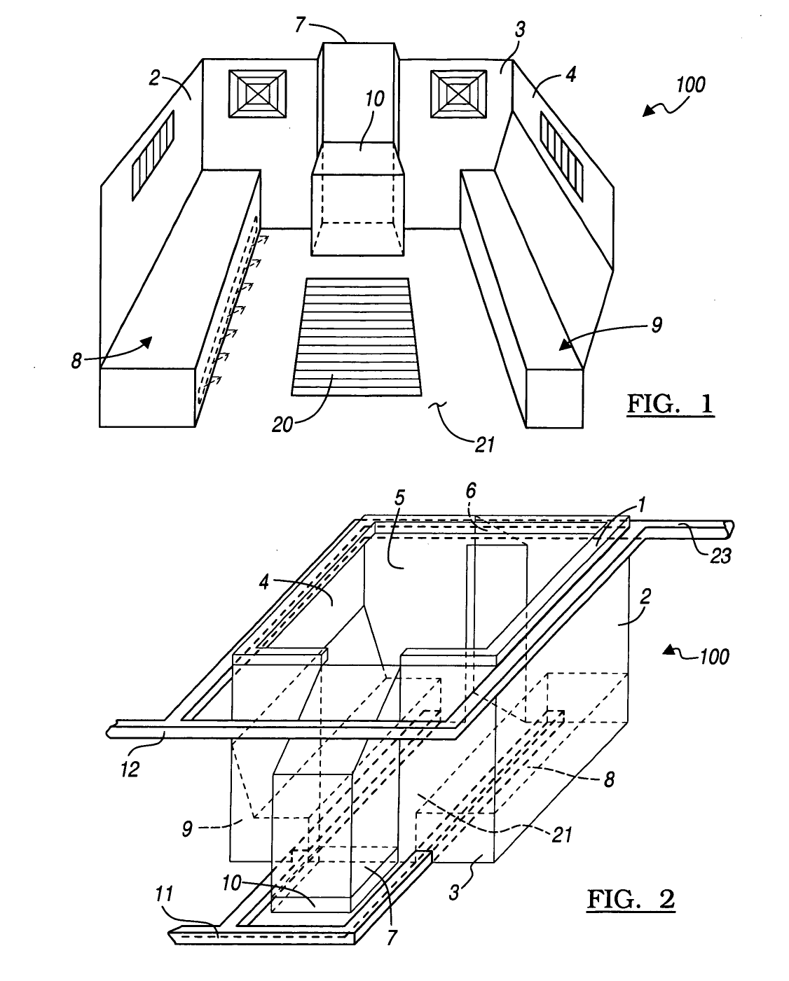

Referring now to FIGS. 1 and 2, room 100 includes one or more treatment areas 8, 9 configured to receive and support occupants of the therapeutic room. Ventilation system includes one or more ventilation passageways 11 provided adjacent the floor 21 to remove sated air from the room 100. One or more ventilation ducts 12 extend about an upper portion of room 100 adjacent the ceiling 1 to provide oxygen rich fresh air into the room 100. Ductwork 23 extending about the outer portions of room 100 separate electrical cords from the heating elements. These cords connect one or more thermostats which measure the conditions in the room to a control panel. Therapeutic rooms 100 are designed to accommodate a variety of therapeutic treatments, including, but not limited to Russian banya, Turkish bath or Finnish sauna style treatments.

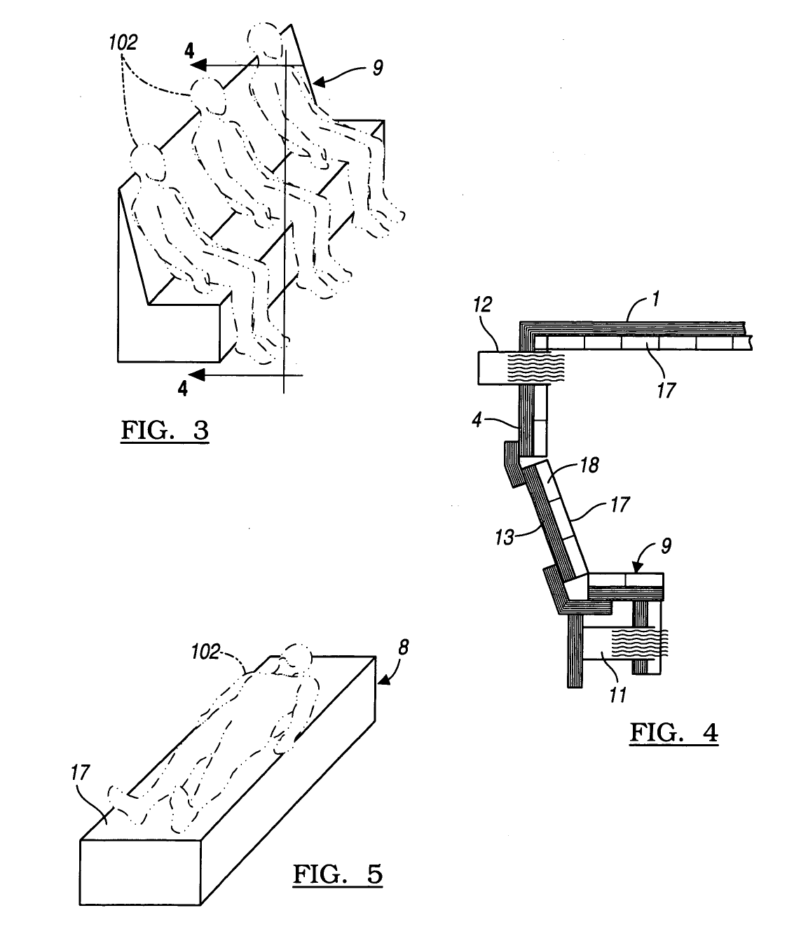

Referring additionally now to FIGS. 3-4, seating bench 9 in the treatment area of room 100 is configured to support one or more occupants 102 in a generally upright position. Seating bench 9 includes a metal support frame 13 supporting one or more heating panels 17 which generate radiant heat. The seating bench 9 is preferably mounted adjacent a sidewall 4 of room 100. However, it is understood that bench 9 may be positioned in a variety of locations in room 100. An exhaust ventilation passage 11 extends through a lower portion of support frame 13. Sidewalls 18 of bench 9 typically are not equipped with heating panels. However, the sidewalls may include heating panels if necessary. It is also understood that the type of materials used to construct the bench, quantities of benches and the size of the bench may vary based on the optimal dimensions selected for the room and the desired number of occupants.

FIG. 5 illustrates a warming table or laying bench 8 provided in the treatment area of room 100. Laying bench 8 is configured to support an occupant 102 in a generally horizontal position. Laying bench 8 is of similar construction to the seating bench 9 and includes a metal support frame supporting one or more heating panels 17 thereon. It is understood that the quantity of benches and dimensions of laying bench 8 may be adjusted based on the dimensions selected for the room.

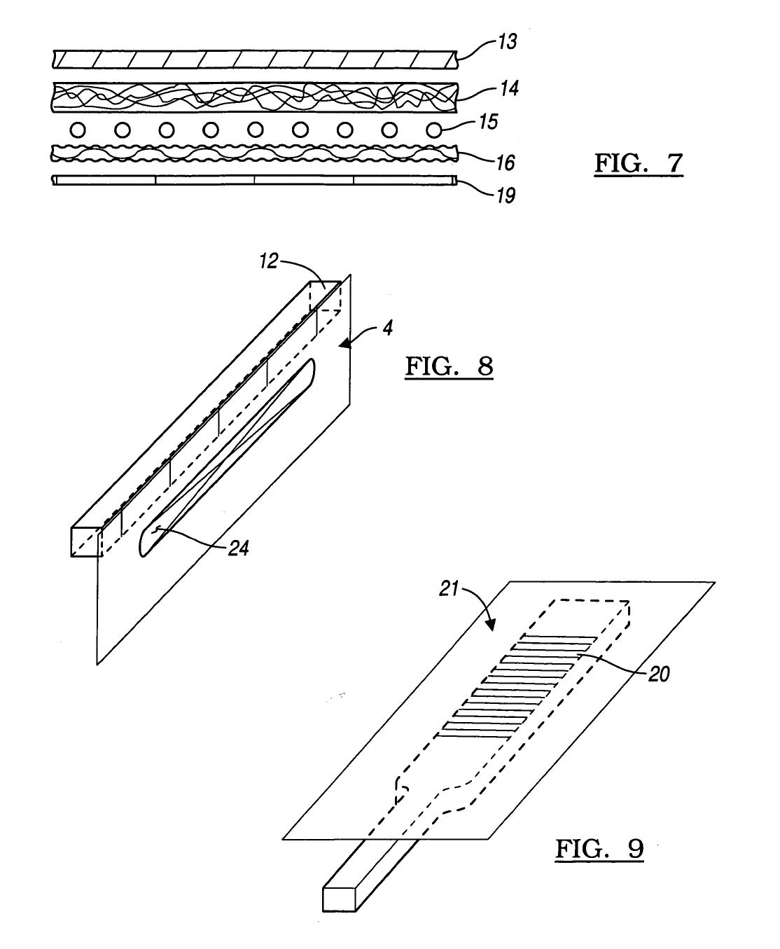

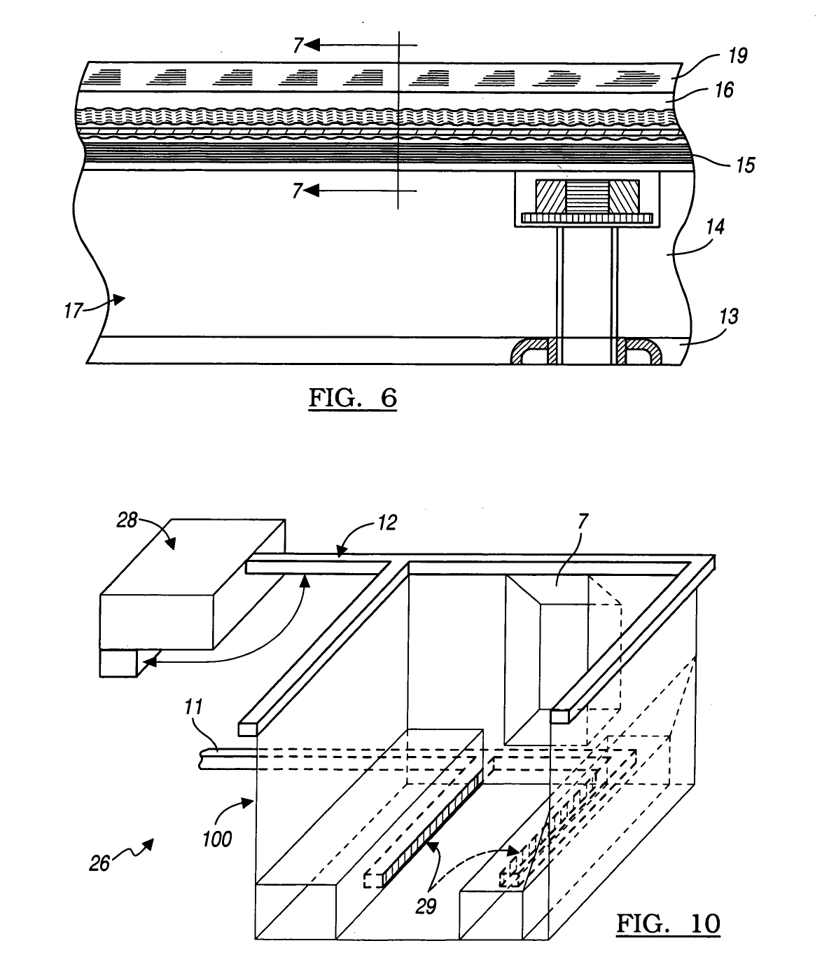

Referring now to FIGS. 6 and 7, the heating panels 17 used in the therapeutic treatment enclosure or room 100 of the present invention are described in greater detail. The heating plates are primarily used on the seating and laying benches in the treatment area. In a preferred aspect of the present invention, panels 17 are provided on all surfaces of the therapeutic treatment room as illustrated in FIG. 4.

Each panel 17 includes a metal support frame or substructure 13 receiving one or more coats of insulation 14. Insulation 14 may be of any suitable variety and configuration. One or more heating elements 15 are disposed adjacent the insulation 14 and embedded in one side of a cement layer 16. The opposing side of cement layer 16 receives one or more heating plates 19. Heating plates 19 are preferably formed of igneous plutonic rock, such as granite. Plates 19 cooperate with elements 15 to generate radiant heat and thereby electromagnetic waves.

Igneous plutonic rock, formed from melted volcanic lava, is preferred for its suitable physical properties. It is understood that other materials may be used in a variety of configurations to accomplish the same objective. Granite exhibits an abrupt decrease in electrical conductivity in the range of 200°-330° Celsius. Thus, granite is well suited for producing “soft” infrared radiant heat electromagnetic waves. These type of waves are generally longer than visible light waves. Longer electromagnetic waves are desirable because they have less energy, thus providing medicinal aspect acts to increase bodily warming without causing chemical or biological change.

The thermal stability and sanitary properties of granite are also desirable, as it is highly heat resistant and does not harbor bacteria. Granite has almost negligible porosity ranging between 0.2 to 4%. Thus, if steam is introduced into the thermal therapeutic room to generate a Russian banya or Finnish sauna style treatment, granite is the best stone to accommodate the added moisture content of the air without affecting the stone.

Heating elements 15 are preferably formed as mineral insulated heating cables. Elements 15 are embedded in the cement or mortar support structure 16 or may be provided below or through the granite overlay. The heating cables provide uniform heat and are formed of either one or two conductor resistance heating wires embedded in highly compressed magnesium oxide, covered by either copper or alloy 825 stainless steel sheath. The mineral insulated cables provide constant wattage output along heated length, for example, a specific wattage output up to 35 watts per lineal foot for copper sheath and up to 50 watts per lineal foot for stainless steel sheath and voltage between 12 and 600 watts.

The cables are preferably constructed of non-flammable inorganic materials that do not degrade and cannot contribute to or cause an electrical fire. Further, the cables are waterproof and resistant to corrosion. Although the optimum temperature range for thermal procedures is to heat the granite to temperatures of 35-65 degrees Celsius (95-149 degrees Fahrenheit), the mineral insulated cable system would run under separate lines from all surfaces to allow us to safely vary temperatures according to the comfort level of the individual user.

The room 100 may be constructed using standard bricks and building materials, but with modifications to allow for our new way to form “soft” infrared radiation for thermal procedures. The formation of “soft” infrared radiation will occur by heating granite stones by electricity to temperatures of about 35-65 degrees Celsius (95-149 degrees Fahrenheit). Depending on the setting of the room temperature, the heated granite will produce electromagnetic waves of in the range of 8 to 9.7 micrometers, from which the body takes energy, causing the body to enjoy a slight rise in temperature. The thermal treatment procedures are based upon an arrangement of heated granite surfaces to expand the plane of radiation to include all surfaces of the floor, ceiling, walls, and seating.

Given a body temperature of approximately 37 degrees Celsius (98.6 degrees Fahrenheit), the body's natural infrared wave length is approximately equal to 9.3 micrometers, allowing the deepest penetration within the body. At 9.3 micrometer wave lengths, complex chemical oxidation-reduction processes, reactions of splitting and synthesis, the process of growth, the formation and dissolution of bio crystals and other biophysical processes occur naturally because there is optimum body temperature. When “soft” infrared radiant heat with a frequency corresponding to the natural frequency of the organism passes through a body, the organism's natural processes are neither altered nor abated, but rather continue uninterrupted.

The therapeutic system uses the combination of heating plates warm the body internally by using the radiant heat of granite stones. The granite stones are heated by electricity to temperatures of 35-65 degrees Celsius (95-149 degrees Fahrenheit) depending on the setting of the room temperature. The heated granite plates will produce electromagnetic waves of about 8 to 9.7 micrometers, from which the body takes energy, causing the body to enjoy a slight rise in temperature. These radiant wavelengths are longer than that of visible light, thus acting safely to increase warming and improve health without causing chemical or biological change. The composition, form, and dimensions of the surfaces in the room are designed to take into account the distribution and diffraction of soft infrared waves in a closed premises.

Although the maximum medicinal effect from the resultant infrared waves will be achieved when the floor temperature is 36 degrees Celsius (98.6° F.), the ceiling and wall temperatures are 65 degrees Celsius (149° F.), and the surfaces for sitting and lying are 48 degrees Celsius (118° F.), the temperature of all surfaces in the sauna room should not exceed 120 degrees Fahrenheit when a person is in the sauna room. It is equally important that the radiant source of heat surrounds the body uniformly, rather than being blown from various directions producing a draft effect. Radiant heat is therefore preferable to air induced heat.

Referring now to FIG. 8, sidewall 4 of the therapeutic treatment room includes a sauna lamp 24. Sauna lamp 24 cooperates with the heating panels to produce infrared radiation applied to the occupant. It is understood that some heat transfer into the room will also result. As illustrated in the Figure, ventilation duct 12 extends behind sidewall 12.

In another aspect of the present invention, a sauna heater is associated with room to provide optional heat and steam for introduction as part of the therapeutic treatment. During treatment, the room may have an ambient room temperature of about 95° F., but the actual room temperature will vary in practice. Influx of fresh air and outflow of sated air from the room will be via a heat recovery unit, described in greater detail below, at a low speed to reduce the possibility of drafts in the room.

Unlike traditional sauna facilities which use heat from the air in the sauna room to heat the body, therapeutic room maintains an ambient air temperature in the room, allowing the occupants to enjoy lower temperatures and more comfortable conditions than other methods. This treatment minimizes shock to the body and overcomes the shortcomings of traditional sauna applications. Additionally, room can be adapted in minutes to allow the user to simulate traditional sauna applications such as the Finnish sauna, Turkish bath, or Russian banya by using a regular sauna heater. Room still provides uniform body heating from all directions at the body's optimal wavelength even when these other sauna adaptations.

As illustrated in FIGS. 1 and 9, a drainage system 20 is provided in the floor 21 of room 100. Drainage system 20 cooperates with the heating panels 102 in floor 21. Drainage system 20 carries away fluids from the therapeutic treatment room and allows a maintenance staff to wash the room, seating and laying benches after use by occupants.

Referring now to FIG. 10, a ventilation system 26 includes one or more heat recovery ventilator units 28. Each heat recovery ventilator unit 28 includes a heat exchanger core and at least one chamber therethrough. Sated air from the thermal therapy room 100 passes through the chamber in one direction, while fresh outside air will pass through the other chamber. The fresh air is routed into the thermal therapy room.

In a preferred aspect of the present invention, the two air streams never mix, but as they pass each other through side-by-side chambers, heat will be transferred from one airflow to the other through highly conductive aluminum walls of the heat exchange core. The air exhausted from the thermal therapeutic room will be used efficiently to heat incoming fresh air while ensuring adequate oxygen content in the room. A damper is provided to ensure that the humidity level of incoming air is resonant with the humidity level in the thermal therapy room.

In another aspect of the present invention, ultraviolet air treatment and cleaning systems in conjunction with all heating and cooling equipment of the facility, including the heat ventilation recovery unit exhaust systems in each thermal therapy room. The ultraviolet treatment system continuously emits high-intensity ultraviolet (UV) energy to kill mold and airborne bacteria passing by the UV light. This is particularly beneficial for patients with respiratory ailments and is similar to the ultraviolet germicidal irradiation technology has been used for many years in medical and industrial applications.

A heat storing steam heater is used to produce moist steam at lower temperatures. The heat storing steam heater includes a digital interface or control panel which controls the heater. In an alternative aspect of the present invention, the control panel may also control the infrared controls used to create a Turkish, Russian or Finnish sauna-type environment. Each thermal therapy room is preferably designed for four persons in a variety of seating configurations, such as three persons sitting while one person lies down. Maximum medicinal effect from the resultant infra-red waves is achieved when the floor temperature is 36 degrees Celsius (98.6° F.), the ceiling and wall temperatures are 65 degrees Celsius (149° F.), and the surfaces for sitting and lying are 48 degrees Celsius (118° F.). It should be noted that the temperature of all surfaces in the thermal therapy room can be manually adjusted to allow us to safely vary temperatures according to the comfort level of the individual user.

Thermostats 29 are placed adjacent exhaust ventilation ducts 11 and inflow ventilation ducts 12. Thermostats 29 are coupled to a control panel and controller. The controller monitors the temperature of the oxygen rich air pumped into the room 100 as well as the number of times fresh air is introduced into the room. The thermostats 29 monitor the content of oxygen rich air in the room as part of the therapy process. The controller further monitors the seating areas to ensure that the heating panels generate electromagnetic waves between about 8 and 9.7 micrometers.

The method of applying therapeutic treatment to an occupant of an enclosure is described in greater detail. One or more treatment areas, such as a seating bench or laying bench described above, are provided in the therapy enclosure. Each of the treatment benches include one or more heating panels having a frame supporting a heating plate and one or more heating elements disposed between the frame and heating plate. The heating elements warm the heating plates to temperatures of about 35-65 degrees Celsius.

Once heated, the heating plates generate radiant heat in the form of electromagnetic waves in the range of about 8 to 9.7 micrometers. The electromagnetic waves are transmitted through the heating panels to the occupant to effect therapeutic changes in human body temperature. The treatment applied to the occupant is monitored by a controller. The controller also monitors the amount of oxygen introduced into the enclosure and sated air removed from the enclosure by the ventilation system through a series of ducts and passageways.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A system for applying therapeutic treatment to a body of an occupant, the system comprising:
a therapy enclosure;
at least one treatment area provided within the therapy enclosure receiving and supporting one or more occupants;
a ventilation system maintaining environmental settings in the enclosure, the ventilation system including one or more ducts supplying oxygen into the enclosure and one or more passageways removing sated air from the enclosure;
one or more heating panels provided on the at least one treatment area configured to generate radiant heat effecting therapeutic changes in human body temperature; and
a controller monitoring the at least one treatment area and ventilation system to control therapeutic conditions in the enclosure.

2. The system of claim 1 wherein the therapy enclosure further comprises a room having a ceiling, a floor, a pair of opposing sidewalls extending therebetween, a forward wall and an opposing rear wall.

3. The system of claim 1 wherein the heating panels comprise a frame supporting a heating plate and one or more heating elements disposed between the frame and heating plate, wherein the heating elements cooperate with the heating plate to generate radiant heat.

4. The system of claim 3 wherein the heating plate is formed of igneous plutonic rock.

5. The system of claim 4 wherein the heating plate is formed of granite.

6. The system of claim 3 wherein the heating elements warm the heating plates to a temperature in the range of about 35 to 65 degrees Celsius to generate an electromagnetic wave between about 8 to 9.7 micrometers.

7. The system of claim 1 wherein the at least one treatment area further comprises a seating bench having a frame supporting one or more heating panels configured to support one or more occupants in a generally upright position.

8. The system of claim 1 wherein the at least one treatment area further comprises a laying bench having a frame supporting one or more heating panels configured to support one an occupant in a generally horizontal position.

9. The system of claim 1 wherein the ventilation system further therapy room further comprises a heat exchanger cooperating with the one or more ducts to introduce heated air into the enclosure.

10. The system of claim 9 wherein the one or more passageways extracting sated air from the enclosure extend through the at least one seating area.

11. The system of claim 9 wherein the ventilation system is configured to produce heated air for Russian banya, Turkish bath or Finnish sauna therapy treatments.

12. A system for applying therapeutic treatment to an occupant, the system comprising:
a therapy room having a ceiling, a floor, a pair of opposing sidewalls extending therebetween, a forward wall and an opposing rear wall;
one or more heating panels disposed in the therapy room configured to generate radiant heat effecting therapeutic changes in human body temperature, the heating panels including a frame supporting a heating plate and one or more heating elements disposed between the frame and heating plate, wherein the heating elements cooperate with the heating plate to generate radiant heat;
a seating bench having a frame supporting one or more heating panels configured to support one or more occupants in a generally upright position;
a laying bench having a frame supporting one or more heating panels configured to support one an occupant in a generally horizontal position;
a ventilation system maintaining environmental settings in the room, the ventilation system including one or more ducts supplying oxygen into the room and one or more passageways removing sated air from the room; and
a controller monitoring the seating bench, laying bench and ventilation system to control therapeutic conditions in the room.

13. The system of claim 12 wherein the heating plate is formed of igneous plutonic rock.

14. The system of claim 13 wherein the heating plate is formed of granite.

15. The system of claim 12 wherein the heating elements warm the heating plates to a temperature in the range of about 35 to 65 degrees Celsius to generate an electromagnetic wave between about 8 to 9.7 micrometers.

16. The system of claim 12 wherein the ventilation system further therapy room further comprises a heat exchanger cooperating with the one or more ducts to introduce heated air into the enclosure.

17. The system of claim 16 wherein the ventilation system is configured to produce heated air for Russian banya, Turkish bath or Finnish sauna therapy treatments.

18. A method of applying therapeutic treatment to an occupant of an enclosure, the method comprising:
providing one or more treatments areas in the enclosure, each treatment area having one or more heating panels having a frame supporting a heating plate and one or more heating elements disposed between the frame and heating plate;
warming the heating plate with the one or more heating elements to temperatures of about 35-65 degrees Celsius;
generating radiant heat in the form electromagnetic waves with the heating plate of about 8 to 9.7 micrometers;
transmitting the electromagnetic waves through the heating panels to the occupant to effect therapeutic changes in human body temperature; and
controlling the radiant heat applied to an occupant of the enclosure.

19. The method of claim 18 wherein the step of providing one or more treatment areas further comprises providing a seating bench for supporting one or more occupants in a generally upright position and a laying supporting an occupant in a generally horizontal position in the enclosure.

20. The method of claim 18 further comprising the step of providing a ventilation system for maintaining environmental settings in the enclosure, wherein the ventilation system including one or more ducts supplying oxygen into the room and one or more passageways removing sated air from the room.
